(19) United States
(12) Patent Application Publication
(10) Pub. No.: US 2014/0357688 A1
(43) Pub. Date: Dec. 4, 2014

(54) METHOD FOR INHIBITING EZH2 EXPRESSION IN BREAST CANCER CELLS

(71) Applicant: Yung Shin Pharm. Ind. Co., Ltd., Tachia (TW)

(72) Inventors: Sheng-Chu Kuo, Taichung City (TW); Fang-Yu Lee, Taichung City (TW); Che-Ming Teng, Taipei City (TW); Ling-Chu Chang, Taichung City (TW); Yung-Luen Yu, Taichung City (TW)

(73) Assignee: Yung Shin Pharm. Ind. Co., Ltd., Tachia (TW)

(21) Appl. No.: 14/289,428

(22) Filed: May 28, 2014

Related U.S. Application Data
(60) Provisional application No. 61/829,066, filed on May 30, 2013.

(51) Int. Cl. A61K 31/416; A61K 9/00

(52) U.S. Cl. CPC A61K 31/416; A61K 9/0053; A61K 9/0019
USPC 514/406; 435/375

(57) ABSTRACT

The present invention is directed to a method for inhibiting the overexpression of EZH2 in breast cancer cells. The method comprises administering to breast cancer cells an effective amount of YC-1 (3-(5′-hydroxymethyl-2′-furyl)-1-benzyl indazole), YC-1-succinate (succinic acid mono-[5-(1-benzyl-1H-indazol-3-yl)-furan-2-ylmethyl]ester), or a pharmaceutically acceptable salt thereof. The present invention is also directed to treating breast cancer comprising administering to a subject an effective amount of YC-1-succinate.

This application claims the benefit of U.S. Provisional Application No. 61/829,066, filed May 30, 2013; which is incorporated herein by reference in its entirety.

### TECHNICAL FIELD

The present invention relates to a method for inhibiting the overexpression of EZH2 in breast cancer cells and a method for treating breast cancer, by administering YC-1 (3-(5′-hydroxymethyl-2′-furyl)-1-benzyl indazole), or YC-1-succinate (succinic acid mono-[5-(1-benzyl-1H-indazol-3-yl)-furan-2-ylmethyl]ester).

### BACKGROUND OF THE INVENTION

Breast cancer is the first common malignancy and second cause of mortality in woman (CA Cancer J Clin 2013, 63:11-30). Basic therapeutic standards involve radiation, surgery, and chemotherapy. A variety of therapeutic targets were identified and valid. The best well known targets of them are estrogen receptor (ER), progesterone receptor (PR), and Her2/Neu. However, about 15-20% breast cancer patients present no effective response or less sensitivity to hormone-based therapies due to the lack of ER, PR, and Her2, which called triple negative breast cancer (TNBC) (Cancer 2012, 118:5463-72). Clinical histopathology diagnosis classified TNBC into the aggressive histological subtype with highly metastatic property (Clin Cancer Res 2004, 10:5367-74). So far, there are no specific therapeutic targets available for the treatment of TNBC. With limited treatment options, TNBC is poor prognosis, high recurrence, and low survival rate (Ann Oncol 2009, 20:1913-27). Therefore, identification of novel therapeutic target for TNBC is urgently needed.

Epigenetic dysregulation plays a critical role in cancer initiation and progression (Mol Cancer Ther 2009, 8:1409-20). The Polycomb group (PcG) proteins are the important epigenetic regulators that mainly function in the silencing of cancer suppress genes and allow to creating advantaged environment for cancer cell growth. They are also involved in cell cycle aberration to benefit stem cell renewal and cancer cell transformation (Stem Cells 2007; 25:2498-510). Dysregulation of PcG proteins can contribute to cancer onset and pathogenesis (Stem Cells 2007, 25:2498-510). PcG proteins are divided into two groups: Polycomb repressive complex (PRC) 1 and PRC2. PRC2 is responsible for initiation of gene silencing, containing enhancer of zeste homolog 2 (EZH2)/EZH1, suppressor of zeste 12 protein homolog (SUZ12), embryonic ectoderm development (EED), and retinoblastoma-binding protein p48 (RbAp48). PRC1 functions as complex with PRC2 to anchor on target chromosome of gene silencing. PRC1 is mainly composed of Ring1A, Ring1B, and Bmi1 (Stem Cells 2007, 25:2498-510). Among PcG proteins, EZH2 is the catalytic unit and severs as histone methyltransferase that trimethylates histone 3 at lysine 27 residue (H3K27me3) to mediate the silence expression of EZH2-targeted genes (Science 2002, 298:1039-43). Overexpression of EZH2 has been reported in a variety of malignancies, including breast cancer, prostate cancer, colon cancer, renal cell cancer as well as hematopoietic malignancies (J Clin Onco 2006, 24:268-73, 8; Clin Cancer Res 2006, 12:1168-74). In breast cells, EZH2 exerts oncogenic properties that are highly associated with cell proliferation, invasion, metastasis, and tumor aggressiveness (Mod Pathol 2011, 24:786-93) and suppression of EZH2 leads to inhibition of metastasis of breast cancer cells (Breast Cancer Res Treat 2012, 131:65-73; Oncogene 2009, 28:843-53). Therefore, EZH2 is regarded as a marker for detection of disease progression and prediction of prognosis after therapy regimens in breast cancer (Proc Natl Acad Sci USA 2003, 100:11606-11; Mod Pathol 2011, 24:786-93).

### SUMMARY OF INVENTION

The present invention is directed to a method for treating breast cancer. The method comprises administering to a subject suffering from breast cancer an effective amount of succinic acid mono-[5-(1-benzyl-1H-indazol-3-yl)-furan-2-ylmethyl]ester (YC-1-succinate) or a pharmaceutically acceptable salt thereof.

The present invention is directed to a method for inhibiting the overexpression of EZH2 in breast cancer cells, comprising administering to the cancer cells an effective amount of YC-1 (3-(5′-hydroxymethyl-2′-furyl)-1-benzyl indazole), YC-1-succinate, or a pharmaceutically acceptable salt thereof.

### BRIEF DESCRIPTION OF THE DRAWINGS

FIG. 1 shows the effect of YC-1 on cell viability of human MDA-MB-468, 184A1, and MCF-10A cells. (A) Cells were treated with indicated concentration of YC-1 for 6, 24, and 48 h. Cell viability was determined by MTT method. (B) MDA-MB-468 cells were treated with 3 μM YC-1 for 0, 6, and 12 h, and then stained with fluorescence diacetate (FDA) and propidium iodide (PI). Cell morphology was assessed by fluorescence microscopy with magnification ×20 (upper 3 rows) or ×40 (lowest row). (C) MDA-MB-468 cells were treated with 3 μM YC-1 for 24 h, cells were lysed and subjected to western blot analysis described in Materials and Methods. β-actin is a loading control. Blots were representative of results from three independent experiments. (D) Cells were treated with vehicle (DMSO, as control) or 3 μM YC-1 for 6 h. The cell cycles were analyzed by flow cytometry. (E) Cells were treated with indicated concentration of YC-1 and then performed with clonogenic assay.

FIG. 2 shows the effect of YC-1 and YC-1-succinate on antitumor activity in MDA-MB-468 xenograft mouse model. MDA-MB-468 cells were used to inoculate Nu/Nu mice. (A, left) Tumor bearing mice were given vehicle (10 μl DMSO, CTL) or YC-1 (30 and 60 mg/kg/day) by ip treatment. (B, right) Tumor bearing mice were given vehicle (normal saline, CTL) or YC-1-succinate (YC-1-S; 20, 40, and 80 mg/kg/day) by oral administration. During treatment period, tumor volume and body weights of mice were measured once per 3 days. Data are expressed as mean of tumor volume (mm³)±S.E.M. from 6 mice (YC-1 treatment) or 10 mice (YC-1-S treatment). Tumor weights (B) and body weights of mice (C) in control and YC-1/YC-1-S-treated groups were recorded.

FIG. 3 shows that YC-1 suppressed EZH2 expression in MDA-MB-468 breast cancer cells. (A)(B) Cells were treated with indicated concentration of YC-1 for 24 h (A) or 3 μM of YC-1 for indicated time (B). Cells were harvested and performed with western blotting analysis. Equal loading was demonstrated by the similar intensities of β-actin. The levels of protein expression were quantitated and shown under blots. (C) Cells were transfected with EZH2 shRNA for 0, 1, 2, 3, and 4 days and collected for determination of cell viability (left) and protein expression (right). (D)(E) After 4 days of EZH2 knockdown, cells were incubated with 3 μM YC-1 for 4 h and detected cell viability (D) and protein contents (E). (F) Tumor specimens were isolated and protein was extracted for western blot analysis.

FIG. 4 shows that YC-1 enhanced proteasome-dependent EZH2 degradation and ubiquitination in MDA-MB-468 breast cancer cells. (A) Cells were pretreated with cyclohexamide (30 μM) for 1 h and followed by induction with vehicle (DMSO) or YC-1 (3 μM) for indicated time. Cells were harvested for detection of protein expression. (B)(C) Cells were incubated with 3 μM YC-1 for 6 h in the presence of vehicle, 3 μM MG-132 or 30 μM NH₄Cl. Cells were harvested for detection of protein expression (B) or cell viability assay (C). The levels of protein expression were quantitated and shown under blots. (D) After treatment of 3 μM YC-1 for indicated time, cells were harvested and lysed for immunoprecipitation and western blot analysis described in Materials and Methods.

FIG. 5 shows the involvement of PKA and ERK in YC-1-induced EZH2 inhibition in MDA-MB-468 breast cancer cells. (A) Cells were pretreated with KT5720 (3 μM), KT5823 (3 μM), NS2028 (30 μM), or ODQ (30 μM) and then stimulated with YC-1 (3 μM) for 6 h. Cells were collected for measurement of protein expression (left) or cell viability (middle). After 3 days of PKA knockdown, cells were induced with 3 μM YC-1 for 4 h. Cells were harvested and analyzed using western blotting (right). The levels of protein expression were quantitated and shown under blots. (B) Cells were preincubated with DMSO (as control), PD98059 (10 μM), SB203580 (10 μM), or SP600125 (10 μM) and followed by induction of 3 μM YC-1 for 6 h. Cells were collected and analyzed protein expression (left) and cells viability (middle). p44/42 MAPK knockdown cells were treated with 3 μM YC-1 for 4 h and then detected protein expression (left). (C) Cells were treated with 3 μM YC-1 for indicated time. Cells were harvested for detection of protein phosphorylated activation. (D) Cells were treated with MG-132 (3 μM), PD98059 (10 μM), or KT5720 (3 μM) for 1 h prior to 3 μM YC-1 treatment. Six hours later, cells were harvested for EZH2 ubiquitination assay. (E) Cells were induced by 3 μM YC-1 for 6 h in the presence of PD98059 or KT5720. Cells were collected for the determination of ERK phosphorylation.

FIG. 6 shows the effect of YC-1 on Src and Raf-1 pathway in MDA-MB-468 breast cancer cells. (A) Cells were pretreated with DMSO (as control), Bay-43-9006 (Bay, 10 μM), farnyesyl thiosalicylic acid (FTS, 10 μM), Src inhibitor I (SrcI, 10 μM), and genistein (Gen, 10 μM), then followed by YC-1 (3 μM) incubation for 6 h. Cells were harvested and lysed for western blot analysis (left, right) and cell viability (middle). The levels of protein expression were quantitated and shown under blots. (B) MDA-MB-468 cells were transfected with shRNA of Raf-1 or Src. After stimulation of 3 μM YC-1 for 4 h, cell protein was subjected to western blot analysis. (C) Cells were incubated with 3 μM YC-1 for indicated time and then lysed for detection of Src, Raf-1, and MEK activation by using specific anti-phosphorylated antibodies. (D) Cells were treated with 3 μM YC-1 for indicated time. Cells were lysed and detected Ras activation by using Raf-RBD conjugated agarose to pull-down Ras-GTP. The total Ras in cell lysate was also detected. (E) Cells were incubated in 3 μM YC-1 for 6 h with or without EGFR inhibitors, AG1478 (AG, 10 μM) or gefitinib (Gef, 10 μM). Cells were lysed and protein expression was determined by western blot analysis. (F) Cells were induced by 3 μM YC-1 in the presence of indicated inhibitors. Cells were lysed and subjected to EZH2 ubiquitination assay.

FIG. 7 shows the effect of YC-1 on Cbl activation in MDA-MB-468 cells. (A) c-Cbl knockdown MDA-MB-468 cells were treated with 3 μM YC-1 for 4 h. Cell were harvested for detection of protein expression. (B) Cells were treated with 3 μM YC-1 for indicated time. Cells were collected and c-Cbl phosphorylation was detected. (C) Cells were pretreated with MG-132 (3 μM) or NH₄Cl (30 μM) for 1 h followed by 3 μM YC-1 induction for 6 h. (D) Cells were pretreated with MG-132 and followed by 3 μM YC-1 induction for indicated time. Cells were harvested and lysed for immunoprecipitation assay. The association between c-Cbl and EZH2 were determined by western blotting analysis and probed with anti-EZH2 or anti-c-Cbl antibody. Membranes were stripped and reprobed with anti-c-Cbl or anti-EZH2 antibody to check the input. (E) Cells were preincubated with or without PD98059 (10 μM) in the presence of MG-132 for 1 h prior to 2 h-treatment of 3 μM YC-1. Then cells were collected and lysate protein was subjected for the detection of the c-Cbl-EZH2 complexes.

FIG. 8 shows the effect of YC-1 on EZH2 mRNA abundance in MDA-MB-468 cells. Cells were treated with 3 μM YC-1 for 3 or 6 h. Cells were collected for the detection of EZH2 mRNA expression with quantitative real-time RT-PCR described in Materials and Methods.

FIG. 9 shows the effect of YC-1 in EZH2 protein expression under normoxia and hypoxia. (A) MDA-MB-468 cells were incubated with indicated concentration of YC-1 for 24 h under normoxia and hypoxia. Cells were collected for assessment of cell viability by MTT assay. (B) Cells were stimulated with vehicle (DMSO, as control), 1 or 3 μM YC-1 for 9 h under normoxia and hypoxia. Cells were harvested and subjected to western blot analysis for the determination of EZH2, HIF-1α, and β-acitn protein expression.

FIG. 10 shows that YC-1 was more sensitive in conducting cell viability and EZH2 inhibition on MDA-MB-468 cells. (A) Cells were incubated with vehicle (DMSO, as control), YC-1 (1, 3 μM) or DZNep (3, 10, 30 μM) for 24 h. Cells were collected and followed the detection of EZH2 protein by western blot analysis. (B) Cells were treated with vehicle, YC-1, and DZNep for 24 h and harvested for the measurement of cell viability.

FIG. 11 shows that YC-1 induced EGFR phosphorylation and suppression in MDA-MB-468 cells. Cells were incubated with 3 μM YC-1 for indicated time. Cells were harvested and subjected to western blot analysis for the detection of protein expression.

FIG. 12 shows that the activation of Akt and CDK1 were not involved in YC-1-downregulated-EZH2 expression. (A) MDA-MB-468 cells were incubated with 3 μM YC-1 for indicated time. Cells were harvested for the detection of phospho-Akt (Ser473), Akt, and PCNA. (B) Cells were pretreated with LY294002 (LY, 10 μM) for 1 h followed by 3 μM YC-1 induction for 6 h. (C) MDA-MB-468 cells were incubated with 3 μM YC-1 for indicated time. Cells were harvested for the detection of phospho-CDK1 (Thr161), CDK1, phospho-EZH2 (Tyr487), EZH2, and β-actin expression with western blot analysis. Levels of EZH2 phosphorylation were calculated by phospho-EZH2 normalized by total EZH2. (D) Cells were treated with YC-1 for 6 h in the absence or presence of roscovitine (Rosc, 20 μM). Cells were harvested and analyzed by western blotting for protein expression. (E) CDK1 knockdown of MDA-MB-468 cells were induced with 3 μM YC-1 for 4 h and followed by western blot analysis.

### DETAILED DESCRIPTION OF THE INVENTION

The present invention is directed to a method for inhibiting the overexpression of EZH2 in breast cancer cells, both in vivo and in vitro. The method comprises administering to breast cancer cells an effective amount of YC-1 (3-(5′-hydroxymethyl-2′-furyl)-1-benzyl indazole), YC-1-succinate (succinic acid mono-[5-(1-benzyl-1H-indazol-3-yl)-furan-2-ylmethyl]ester), or a pharmaceutically acceptable salt thereof. “An effective amount” is an amount effective to inhibit the overexpression of EZH2 in breast cancer cells.

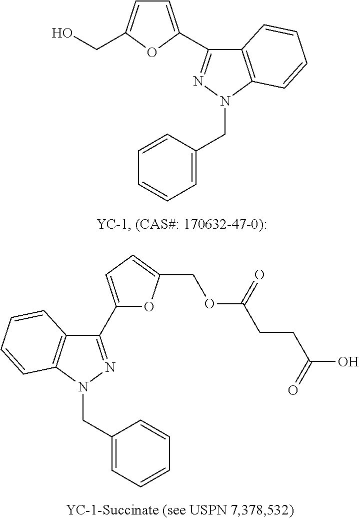 [SMILES: OCc1ccc(-c2nn(Cc3ccccc3)c3ccccc23)o1 ; O=C(O)CCC(=O)OCc1ccc(-c2nn(Cc3ccccc3)c3ccccc23)o1]

EZH2, a histone trimethyltransferase, is overexpressed by cancer cells and functions as a tumor suppressor gene of epigenetic silencing, and its expression level is highly correlated to cancer metastasis ability. The inventors have discovered that YC-1 and YC-1-succinate act as an inhibitor of EZH2, particularly in breast cancer cells. Administering YC-1 or YC-1-succinate to breast cancer cells is effective in reducing tumor size and tumor weight. The inventors have demonstrated that YC-1 inhibited cell viability and clonogenic ability and enhance caspases activation in breast cancer cells. The inventors have also shown that YC-1 reduced tumor growth in MDA-MB-468 xenograft mouse model. YC-1 concentration- and time-dependently downregulated the expression of EZH2 as well as other Polycomb repress complex members, including SUZ12, RbAp48, Ring1A, Ring1B, and Bmi1. Knockdown of EZH2 reduced the susceptibility of MDA-MB-468 cells to YC-1-induced apoptosis. Proteasome inhibitor, MG-132, modulated YC-1-induced-EZH2 inhibition. Both degradation rate and ubiquitination of EZH2 protein were enhanced by YC-1. Down-regulation of EZH2 by YC-1 was associated with the activation of protein kinase A and Src-Raf-ERK-mediated pathways. The inventors have discovered that YC-1 induces apoptosis and inhibition of tumor cell growth on MDA-MB-468 breast cancer cells through a down-regulation mechanism of EZH2 by activating c-Cbl and ERK.

The present invention is also directed to a method for treating breast cancer, comprising administering to a subject suffering from breast cancer an effective amount of YC-1 or YC-1-succinate, or a pharmaceutically acceptable salt thereof. “An effective amount,” is the amount effective to treat breast cancer.

### Pharmaceutical Compositions

YC-1 or YC-1-succinate, which is the active ingredient of the present invention, can be used directly as a pharmaceutical composition. YC-1 or YC-1-succinate can also be formulated in a pharmaceutical composition which comprises YC-1 or YC-1-succinate and one or more pharmaceutically acceptable carriers. The pharmaceutical composition can be in the form of a liquid, a solid, or a semi-solid.

Pharmaceutically acceptable carriers can be selected by those skilled in the art using conventional criteria. Pharmaceutically acceptable carriers include, but are not limited to, sterile water or saline solution, aqueous electrolyte solutions, isotonicity modifiers, water polyethers such as polyethylene glycol, polyvinyls such as polyvinyl alcohol and povidone, cellulose derivatives such as methylcellulose and hydroxypropyl methylcellulose, polymers of acrylic acid such as carboxypolymethylene gel, nanoparticles, polysaccharides such as dextrans, and glycosaminoglycans such as sodium hyaluronate and salts such as sodium chloride and potassium chloride.

The pharmaceutical composition of this invention can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique. Preferred routes of administration are oral administration and intravenous injection.

A sterile injectable composition can be a solution or suspension in a non-toxic parenterally acceptable diluent or solvent, such as a solution in 1,3-butanediol. Among the acceptable vehicles and solvents that can be employed are mannitol and water. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For example, a tablet formulation or a capsule formulation may contain other excipients that have no bioactivity and no reaction with rhamnolipids. Excipients of a tablet may include fillers, binders, lubricants and glidants, disintegrators, wetting agents, and release rate modifiers. Binders promote the adhesion of particles of the formulation and are important for a tablet formulation. Examples of binders include, but not limited to, carboxymethylcellulose, cellulose, ethylcellulose, hydroxypropylmethylcellulose, methylcellulose, karaya gum, starch, corn starch, and tragacanth gum, poly(acrylic acid), and polyvinylpyrrolidone. A tablet formulation may contain 1-90% of YC-1 or YC-1-succinate. A capsule formulation may contain 1-100% of YC-1 or YC-1-succinate.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art. A composition having YC-1 and YC-1-succinate can also be administered in the form of suppositories for rectal administration.

In another embodiment, the pharmaceutical composition comprises one or more YC-1 or YC-1-succinate imbedded in a solid or semi-solid matrix, and is in a liquid, solid, or semi-solid form. The pharmaceutical composition can be injected subcutaneously to a subject and then the active ingredients slowly released in the subject. The formulation may contain 1-90% YC-1 or YC-1-succinate.

The pharmaceutical composition is preferred to be stable at room temperature for at least 6 months, 12 months, preferably 24 months, and more preferably 36 months. Stability, as used herein, means that YC-1 or YC-1-succinate maintains at least 80%, preferably 85%, 90%, or 95% of its initial activity value.

The pharmaceutical compositions of the present invention can be prepared by aseptic technique. The purity levels of all materials used in the preparation preferably exceed 90%.

Dosing of the composition can vary based on the disease state and each patient's individual response. For systemic administration, plasma concentrations of active compounds delivered can vary; but are generally 1×10-10⁻¹×10⁻⁴ moles/liter, and preferably 1×10⁻⁸-1×10⁻⁵ moles/liter.

In one embodiment, the pharmaceutical composition comprising YC-1 or YC-1-succinate is administrated orally to a human subject. The dosage of YC-1 or YC-1-succinate for oral administration is generally 1-10, and preferably 2-8 or 3-6 mg/kg/day. The oral pharmaceutical composition is administered 1-4 times daily, preferably 1-2 times daily.

In one embodiment, the pharmaceutical composition is administrated by intravenous injection to a human subject. The dosage or YC-1 or YC-1-succinate by intravenous injection is 1-10, and preferably 2-8 or 3-6 mg/kg/day.

Those of skill in the art will recognize that a wide variety of delivery mechanisms are also suitable for the present invention.

The present invention is useful in treating a mammalian subject, such as humans, dogs and cats. The present invention is particularly useful in treating humans.

The following examples further illustrate the present invention. These examples are intended merely to be illustrative of the present invention and are not to be construed as being limiting.

### Examples

Abbreviation: Cbl, Castias B-lineage lymphoma; CDK1, cyclin-dependent kinase 1; EED, embryonic ectoderm development; EGFR, epidermal growth factor receptor; ER, estrogen receptor; EZH2, enhancer of zeste homolog 2; HIF-1α, hypoxia-inducible factor-1α; H3K27me3, histone 3 lysine 27 trimethylation; PcG, Polycomb group; PKA, protein kinase A; PKG, protein kinase G; PI3K, Phosphoinositide-3-kinase; PR, progesterone receptor; SUZ12, suppressor of zeste 12 protein homolog; TNBC, triple negative breast cancer

### Materials and Methods

### Reagents and Antibodies

YC-1 and YC-1-succinate was obtained from Yung-Shin Pharmaceutical Industry Co. Ltd. (Taichung, Taiwan). DMEM/F12 medium, RPMI-1640 medium, fetal bovine serum (FBS), penicillin, and streptomycin were purchased from Hyclone Laboratories (Logan, Utah, USA). Inhibitors: PD98059, SB203580, SP600125, MG-132, KT5720, KT5823, NS2028, Bay-43-9006, farnyesyl thiosalicylic acid, manumycin A, DZNep, roscovitine, and AG1478 were obtained from Cayman (Ann Arbor, Mich., USA). Src Kinase Inhibitor I and LY294002 were purchased from EMD Millipore Corporation (Billerica, Mass., USA). Cycloheximide, ODQ, genistein, and gefitinib were obtained from Sigma-Aldrich (St. Louis, Mo., USA). These inhibitors and YC-1 were dissolved in dimethyl sulfoxide (DMSO) and final concentration was less than 0.1% (v/v). Antibodies against EZH2, SUZ12, Ring1A, Ring1B, Bmi1, phospho-p44/42 MAPK (ERK1/2) (Thr202/Tyr204), phospho-MEK1/2 (Ser217/221), phospho-p38 MAPK (Thr180/Tyr182), phospho-Src (Tyr416), phospho-c-Raf (Ser338), phospho-c-Cbl (Tyr731), phospho-c-Cbl (Tyr774), phospho-EGFR (Tyr1045), phospho-EGFR (Tyr1068), EGFR, caspase-3, caspase-8, caspase-9, PARP, p44/42 MAPK (ERK1/2), MEK1/2, Src, p38 MAPK, PKA C-α, PKA RI-α, Tri-Methyl-Histone H3 (Lys27), and c-Cbl were purchased from Cell Signaling Technology (Beverly, Mass., USA). Antibodies against ubiquitin, Ras, and β-actin were from EMD Millipore. Antibodies against PCNA, phospho-Akt (Ser473), Akt, and histone 3 were purchased from Santa Cruz Biotechnology (Santa Cruz, Calif., USA). Antibodies against HIF-1α, EED, RbAp48, phospho-EZH2 (Thr487), α-tubulin, and phospho-CDK1 (Thr161) were obtained from GeneTex (Irvin, Calif., USA). Antibodies against EZH2, Raf-1, CDK1, phospho-JNK (Thr183/Tyr185), and JNK were from BD Biosciences (San Diego, Calif., USA). Other reagents were purchased from Sigma-Aldrich.

### Cell Culture

Human breast cancer cells, MDA-MB-468, MDA-MB-157, MDA-MB-231, MDA-MB-453, and Hs578T, and immortalized mammary epithelial cell lines, 184A1 and MCF-10A, were obtained from American Type Culture Collection (Manassas, Va., USA). MDA-MB-468, MDA-MB-157, MDA-MB-231, and MDA-MB-453 were cultured in DMEM/F12 medium supplemented with 10% FBS, 100 U/ml penicillin, and 100 μg/ml streptomycin. Hs578T was cultured in RPMI 1640 medium containing 10% FBS, 2 mM L-glutamine, 100 U/ml penicillin, and 100 μg/ml streptomycin. 184A1 and MCF-10A were cultured in DMEM/F12 medium supplemented with 5% horse serum, 0.5 μg/ml hydrocortisone, 10 μg/ml insulin, 20 ng/ml epidermal growth factor, 0.1 μg/ml cholera enterotoxin, 2 mM L-glutamine, 100 U/ml penicillin, and 100 μg/ml streptomycin. All cells were maintained in a humidified incubator containing 5% CO₂. For hypoxia treatment, cells were incubated in a chamber flushed with 1% 0₂, 5% CO₂, and 94% N₂ at 37° C.

### Cell Viability Assay

Cell viability was measured by 3-(4,5-Dimethyl-2-thiazolyl)2,5-diphenyl-2H-tetrazolium bromide (MTT) assay.

### Morphological Analysis of Apoptosis by Fluorescent Staining

Vital and apoptotic cells were observed under staining with fluorescein diacetate (2 mg/ml) and propidium iodide (4 mg/ml) for 10 min. Cells were visualized and recorded on a LeicaDC300 microscope with digital camera.

### Colony Formation Assay

After exposure to YC-1, cells were collected and washed extensively with PBS. Five hundred cells per well was seeded onto 6-well plates and maintained in a 37° C., 5% CO₂ incubator. Three weeks later, colonies were fixed with formaldehyde (3.7%, v/v) and stained with crystal violet (0.5%, w/v), then counted.

### Cell Cycle Analysis

Cells (1×10⁶) treated with YC-1 were fixed in cold 70% ethanol overnight. Cells were stained with staining solution (0.5% Triton X-100, 2 mg/ml propidium iodide, 1 mg/ml RNase A in 1×PBS), followed by analysis on a FACSCalibur flow cytometer (BD Biosciences, Mountain View, Calif., USA).

### Western Blot Analysis

Cells were lysed in PBS containing proteinase inhibitor and phosphatase inhibitors and sonication. Protein concentration was measured using the Bio-Rad protein assay kit (Bio-Rad Laboratories, Hercules, Calif., USA). Lysate protein was separated by electrophoresis of SDS-PAGE and transferred to Immobilon P membrane (EMD Millipore). The membranes were incubated with appropriate primary antibodies and horseradish peroxidase-conjugated secondary antibodies (EMD Millipore). The signaling was visualized using Chemiluminescence substrate kit (EMD Millipore) and collected by the Luminesence image analyzer, LAS4000 (Fuji Photo Film Co., Tokyo, Japan). The band intensities were analyzed and quantified by the Multi Gauge software (Fuji Photo Film).

shRNA Transfection and Cell Infection

The pCMV-ΔR8.91, pMD.G, and specific short hairpin-PLKO.1 (shRNA) plasmids were purchased from the National RNAi Core Facility Academia Sinica, Taiwan. shRNA clones used in this study were described in Table 1. Lentivirus particles were produced by transient transfection with specific shRNA and packaging vectors (pCMV-ΔR8.91 and pMD.G) using Lipofectamine 2000 transfection reagent (Invitrogen Corp., Carlsbad, Calif., USA) in 293T cells. Forty-eight hours after transduction, the media were filtered with 0.22 μm filter and used for infection. MDA-MB-468 cells were infected with specific shRNA viral-contained supernatant in the presence of polybrene (8 μg/ml). After 24-h incubation, media were replaced with complete medium containing puromycin (2 μg/ml). Cells were applied for tests and harvested based on the experiment required.

TABLE 1
shRNAs used in this study

| shRNA | Target | Gene | shRNA |
|---|---|---|---|
| Name | Gene | Number | Clones |
| Shc-Cbl#1 | c-Cbl | 867 | TRCN0000039727 |
| Shc-Cbl#2 | c-Cbl | 867 | TRCN0000288694 |
| shCDK1#1 | CDK1 | 983 | TRCN0000000583 |
| shCDK1#2 | CDK1 | 983 | TRCN0000196603 |
| shERK#1 | MAPK1 | 5594 | TRCN0000010039 |
| shERK#2 | MAPK1 | 5594 | TRCN0000195517 |
| shEZH2#1 | EZH2 | 2146 | TRCN0000040076 |
| shEZH2#2 | EZH2 | 2146 | TRCN0000040073 |
| shEZH2#3 | EZH2 | 2146 | TRCN0000010474 |
| shLuc | | | TRCN0000231740 |
| shPKA#1 | PKA catalytic subunit | 5566 | TRCN0000233527 |
| shPKA#2 | PKA catalytic subunit | 5566 | TRCN0000356093 |
| shRaf-1#1 | Raf-1 | 5894 | TRCN0000001065 |
| shRaf-1#2 | Raf-1 | 5894 | TRCN0000001068 |
| shSrc#1 | Src | 6714 | TRCN0000038150 |
| shSrc#2 | Src | 6714 | TRCN0000038153 |

### Ubiquitination Assay

Cells were lysed with MLB buffer (25 mM HEPES, pH 7.5, 150 mM NaCl, 1% Igepal CA-630, 10 mM MgCl₂, 1 mM EDTA, 2% glycerol, 1 mM Na₃VO₄, 1 mM NaF, 1 mM PMSF, 10 μg/ml of leupeptin, aprotinin, and pepstatin A). After sonication, cell lysates were centrifugated at 12,000×g for 10 min at 4° C. Supernatants were incubated with anti-EZH2 antibody (BD Biosciences) and protein A Sepharose beads (GE Health Care) overnight at 4° C. After washing with MLB buffer, precipitated proteins were boiled in 1× Laemmli sample buffer and then separated with SDS-PAGE. The ubiquitination levels of EZH2 were recognized by using anti-Ubiquitn monoclonal antibody.

### Co-Immunoprecipitation

Cells were lysed with RIPA buffer (50 mM Tris-C1, pH 7.5, 1% Igepal CA-630, 150 mM NaCl, 1 mM EDTA, 1 mM Na₃VO₄, 1 mM NaF, 1 mM PMSF, 10 μg/ml of leupeptin, aprotinin, and pepstatin A) for 30 min at 4° C. One milligram of cell lysate was incubated with anti-EZH2 or anti-c-Cbl antibody and protein A-Sepharose beads, then gently rotated at 4° C. overnight. Then, immune complexes were precipitated and subjected to western blot analysis.

### Ras Activation

Ras-GTP has a high affinity with Ras binding domain of Raf-1 (Raf-RBD). Accordingly, GST fusion protein containing Raf-RBD was applied to detect Ras activation. Cells were lysed in MLB buffer and cell protein (500 μg) was incubated with agarose conjugated Raf-RBD (EMD Millipore) overnight at 4° C. The agarose was collected by centrifugation, washed, and re-suspended in 1× Laemmli sample buffer. Samples were boiled and then performed western blot analysis.

### Quantitative Real-Time Reverse Transcription PCR

Total RNA was isolated from MDA-MB-468 cells treated with YC-1 and extracted using TRIzol® Reagent (Invitrogen). cDNA strain was reverse transcribed by oligo dT₍₁₅₎ using M-MLV reverse transcriptase (Invitrogen) according to the manufacturer's instructions. Quantitative real-time RT-PCR analysis was performed by a LightCycler® 480 II RTPCR system (Roche Applied Sciences, Manheim, Germany) using Fast Start DNA Master Plus SYBR Green I kit (Roche Applied Sciences). PCR primers were as follows: human EZH2 (NM_{—}004456.4) 5′-CGCTTTTCTTCTGTAGGCGATGT-3′ (Forward), 5′-TGGGTGTTGCATGAAAAGAAT-3′ (Reverse); human GAPDH (NM_{—}002046.3) 5′-AGCCACATCGCTCAGACAC-3′ (Forward); 5′-GCCCAATACGACCAAATC-3′ (Reverse). The expression level of EZH2 mRNA was normalized against the level of GAPDH mRNA in the same sample.

### MDA-MB-468 Breast Cancer Xenograft Animal Model

Nu/Nu female mice (four weeks old) were from National Laboratory Animal Center, Taipei, Taiwan. Mice were maintained under procedures and guidelines from the Institutional Animal Care and Use of the National Health Research Institutes. MDA-MB-468 breast cancer cells (5×10⁶ cells per mouse) were suspended in 0.1 ml of Matrigel solution (50%, v/v, Matrigel in 1×PBS) and inoculated into the mammary fat pads of nude mice. When the tumor masses reached to 100 mm³, the tumor bearing mice were randomly divided into different groups for different dosage of YC-1 treatments. The mice were given by intraperitoneal injection with YC-1 or oral administration of YC-1-succinate. Tumor size and body weights of mice were measured once per 3 days and tumor volume (mm³) was calculated as the equation: length×(width)×0.5. At the end of experiments, mice were sacrificed and tumor nodules were dissected and weighted. Tumor tissues were subjected to western blot analysis.

### Statistical Analysis

All data are expressed as mean±standard error (S.E.M.) of three independent experiments. Data for statistical difference and means were analyzed using the t-test. p-values less than 0.05 was considered statistically significant (*p<0.05, **p<0.01).

### Results

### Example 1

### Anticancer Activity of YC-1 on MDA-MB-468 Cells

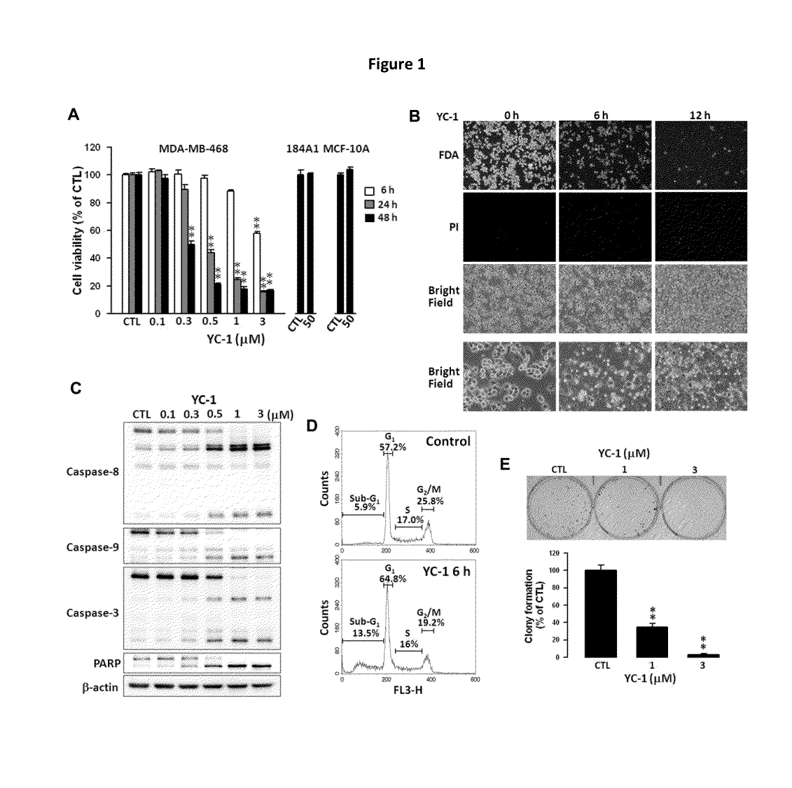

First we investigated the effect of YC-1 on cell viability of MDA-MB-468, a malignant TNBC cells. YC-1 significantly concentration- and time-dependently reduced cell viability of MDA-MB-468, IC₅₀ values were 0.62±0.02 μM and 0.29±0.02 μM at 24 h- and 48 h-incubation respectively, while no effect on the cell viability of normal mammary epithelial cells, 184A1 and MCF-10A (FIG. 1A). As shown in FIG. 1B, obviously morphological changes with apoptotic characteristics were observed after 6 h-treatment of YC-1, including cell shrinkage, blebbing, and DNA break. For evaluation of pro-apoptotic activation exposed to YC-1, we performed western blotting of caspase-8, -9, and -3 and PARP. Data showed that YC-1 clearly caused the cleavage of caspases and PARP (FIG. 1C). Cell cycle distribution was performed to analyze whether YC-1-induced viability inhibition is associated with cell cycle alternation. Treatment of YC-1 slightly increased the percentage of cells at the G₁ phase (57.2% vs 64.8; control vs 6 h post-treatment) whereas significantly increased sub-G₁ population (5.9% vs 13.5%; control vs 6 h post-treatment) (FIG. 1D). Additionally, antitumor capacity of YC-1 was evaluated by clonogenic activity, indicating YC-1 had a potent ability in attenuation of tumor formation (FIG. 1E). Several other triple negative breast cancer cell lines were also under investigation with YC-1 treatment. The results showed slight inhibitory effect on MDA-MB-231 and no effect on MDA-MB-157, MDA-MB-453, and Hs578T.

### Example 2

### Effect of YC-1 and YC-1-Succinate on Antitumor Activity in Xenograft Animal Model

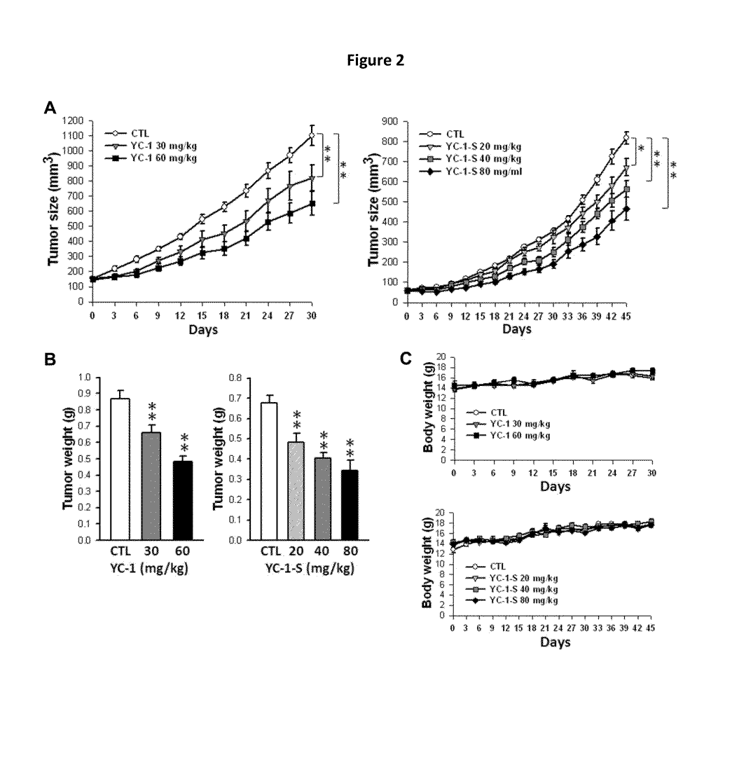

Antitumor activities of YC-1 and YC-1-succinate were investigated in nude mice inoculated with MDA-MB-468 cells. MDA-MB-468 tumor bearing mice were administered with 30 and 60 mg/kg of YC-1 by intraperitoneal injection. As shown in FIG. 2A (left), 30 and 60 mg/kg of YC-1 significantly inhibited the tumor growth. Effect of YC-1's prodrug YC-1-succinate (YC-1-S) in MDA-MB-468 tumor bearing mice was also investigated. Mice were orally administrated with 20, 40, and 80 mg/kg of YC-1-S. In vivo pharmacokinetic analysis revealed that YC-1-S quickly converted into its active form. YC-1-S displayed a dose-dependent inhibition on MDA-MB468 tumor growth (FIG. 2A, right). Both YC-1 and YC-1-S dose-dependently reduced tumor weight (FIG. 2B). Moreover, body weights of mice were not affected by YC-1 and YC-1-S (FIG. 2C).

### Example 3

### YC-1 Downregulates the Expression of EZH2

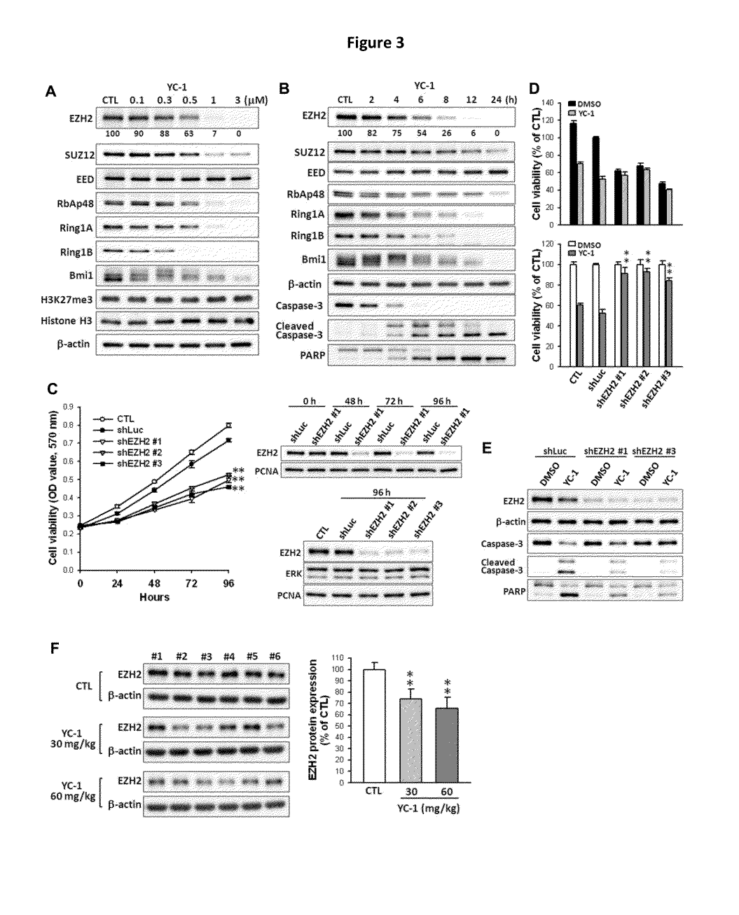

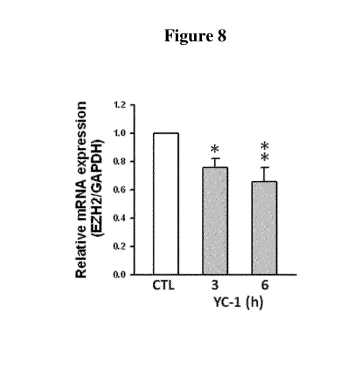

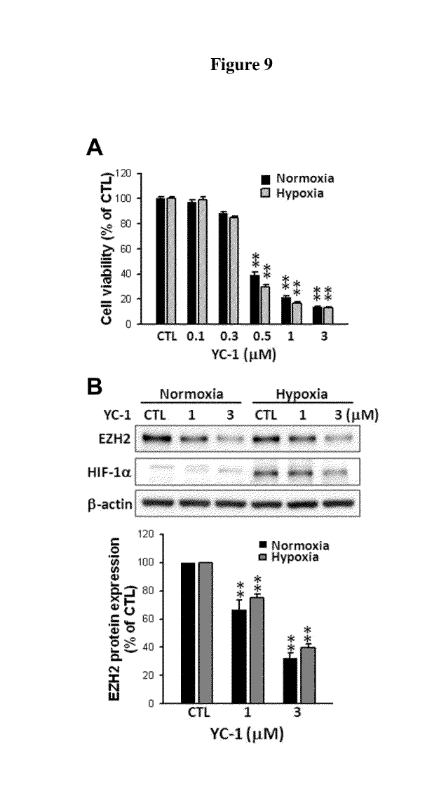

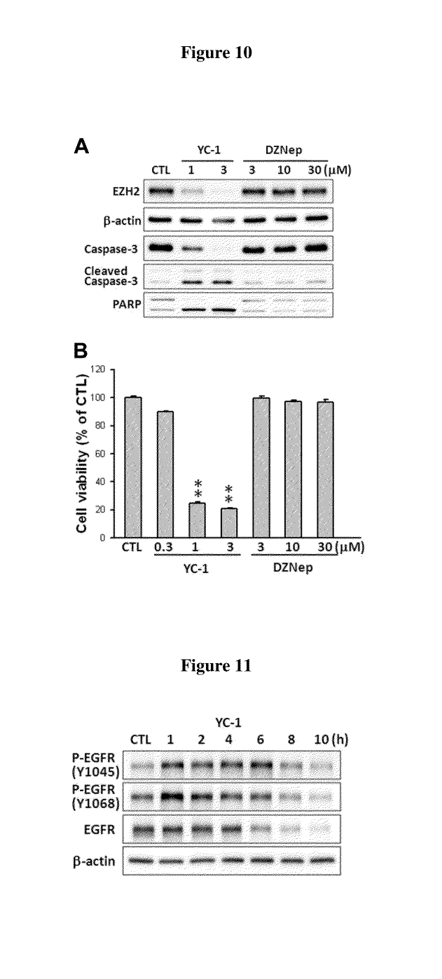

PcG proteins play an important role in breast cancer progression (2, 8, 24). Especially, EZH2 is regarded as a marker of aggressive malignancies, which displays a high association with disease progression (22, 24). We next investigated the effects of YC-1 on PRC1 and PRC2 proteins in vitro. As shown in FIG. 3A, YC-1 displayed an effectively concentration-dependent suppression on PRC2 proteins expression, including EZH2, SUZ12, and RbAp48, but no effect on EED. The IC₅₀ value of YC-1 inhibited EZH2 expression was 0.54±0.04 μM at 24 h-incubation. YC-1 could quickly reduce EZH2 and a significant inhibition were detected at 2 h (about 18% inhibition) (FIG. 3B). Meanwhile, earliest effects of caspase-3 activation were detected at 4 h (FIG. 3B). Treatment of YC-1 also decreased several PRC1 components, including Ring1A, Ring1B, and Bmi1 (FIGS. 3A and 3B). However, H3K27me3, an EZH2 downstream molecule, kept unchanged when exposed to YC-1 (FIG. 2A). Besides, levels of EZH2 gene expression were also checked. YC-1 showed an inhibitory effect on the mRNA expression of EZH2, but less inhibition than protein levels (FIG. 8). EZH2 inhibition by YC-1 was also examined under hypoxia. YC-1 showed the similar inhibition pattern in both cell viability and EZH2 protein expression in normoxia and hypoxia (FIGS. 10A and 10B).

Potency of YC-1 in EZH2 inhibition was compared to a known EZH2 inhibitor, 3-deazaneplanocin A (DZNep) (14, 40). DZNep failed to inhibit EZH2 and activate apoptosis on MDA-MB-468 even with ten folds of concentration higher than YC-1 after 24 h treatment (FIGS. 10A and 10B). To explore whether down-regulation of EZH2 contributes to the cell death of MDA-MB-468, we used lentivirus-mediated specific short hairpin RNA to deplete EZH2 in MDA-MB-468 cells. Cell viability was inhibited following the decrease of EZH2 levels (FIG. 3C). Moreover, knockdown of EZH2 significantly de-sensitized MDA-MB-468 cells in response to YC-1 and less induction in cell death and cleavage of caspase-3 and PARP were observed when compared to control shRNA transfected cells (FIGS. 3D and 3E). These results indicated the inhibition of EZH2 may account for YC-1-induced apoptosis in MDA-MB-468 cells. Furthermore, YC-1 also could decrease EZH2 level in tumor from MDA-MB-468 xenograft mice (FIG. 3F), showing the suppression of EZH2 accounts for the inhibition of tumor growth.

### Example 4

### YC-1 Decreases the Stability of EZH2 and Enhances Proteasome Degradation

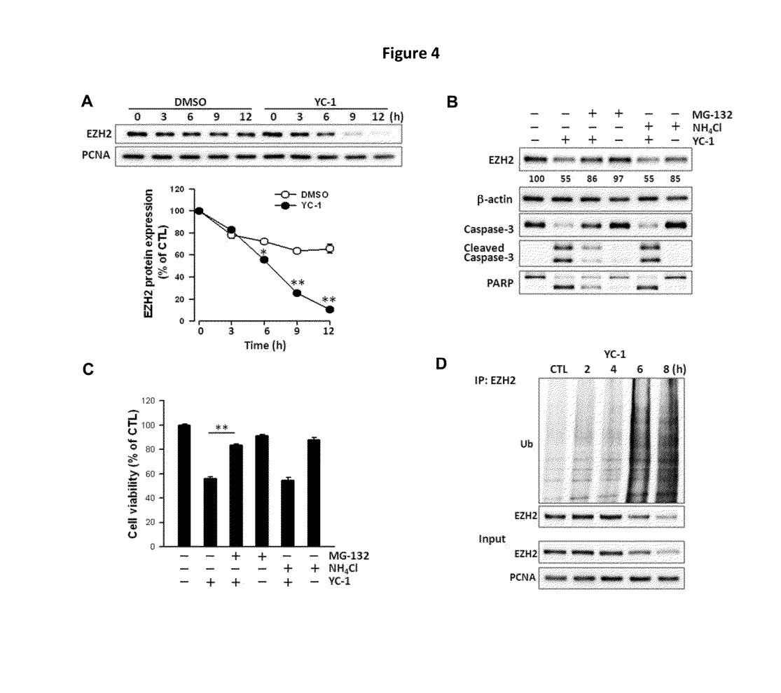

To test the possibility of YC-1 inhibits EZH2 protein expression by enhancing protein degradation, the protein stability of EZH2 was evaluated by YC-1 treatment in the presence of cycloheximide, a protein translation inhibitor. As shown in FIG. 4A, the degradation rate of EZH2 protein was accelerated in cells treated with YC-1 (t_{1/2}=6.6±0.2 h) compared with vehicle-treated cells (t_{1/2}=14.8±0.2 h, data not shown). Pretreatment with proteasome inhibitor (MG-132), but not lysomsome inhibitor (NH₄Cl), significantly prevented EZH2 degradation in response to YC-1 induction (FIG. 4B). Moreover, MG-132 reversed the YC-1-induced inhibition of cell viability (FIG. 4C). YC-1 time-dependently promoted the ubiquitination of EZH2 that is negatively correlated to suppression of EZH2 (FIG. 4D). These results suggested that YC-1 increases EZH2 ubiquitination followed by proteasome degradation.

### Example 5

### Protein Kinase a and ERK are Involved in YC-1-Downregulated EZH2

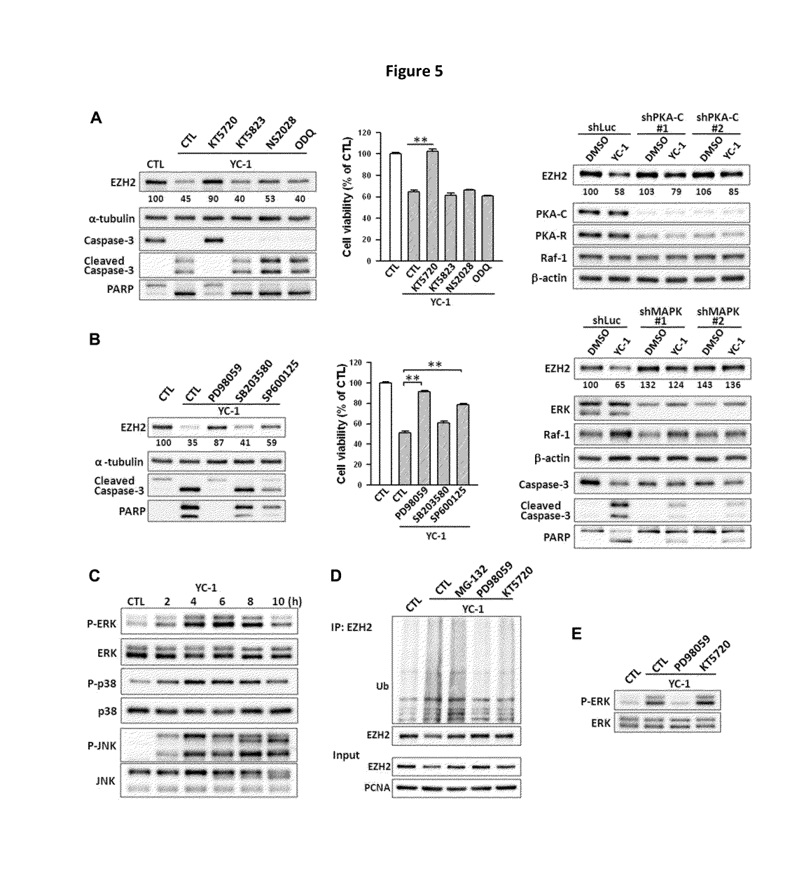

We next tested the possible signaling pathways involved in suppression of EZH2 by YC-1. YC-1 is a well-known cGMP and cAMP activator (Br J Pharmacol 2002; 136:558-67). Therefore, we reasoned that YC-1 may inhibit EZH2 expression through PKG- or Protein kinase A (PKA)-dependent pathway in MDA-MB-468 cells. KT5720 (PKA inhibitor), KT5823 (PKG inhibitor), NS2028 (PKG inhibitor), and ODQ (sGC inhibitor) were applied to investigate the effects of YC-1 on EZH2 expression and cell viability. In contrast to KT5720 significantly reversed the inhibition of YC-1-induced EZH2 expression and cell viability, KT5823, NS2028, and ODQ failed to have effects on YC-1-inhibited EZH2 expression and cell viability (FIG. 5A, left, middle). Furthermore, knockdown of PKA catalytic domain by shRNA attenuated the inhibition of EZH2 by YC-1 (FIG. 5A, right).

That activation of MAPK-related pathways is required for YC-1 to conduct anticancer activity has been proved in cancer cells (Br J Pharmacol 2002; 136:558-67). Specific inhibitors, PD98059 (MEK inhibitor), SB203580 (p38 MAPK inhibitor), and SP600125 (JNK inhibitor) were used to test the role of MAPKs in EZH2 inhibition. Among these inhibitors, PD98058 almost completely abolished YC-1-mediated inhibition in both EZH2 expression and cell viability (FIG. 5B, left, middle). Furthermore, YC-1 failed to suppress EZH2 and induce cell death while ERK protein was depleted (FIG. 5B, right). YC-1 treatment caused a rapid phosphorylated activation of ERK, beginning at 2 h and reaching a maximal activation at 6 h (FIG. 5C). The ubiquitination of EZH2 could be blocked by KT5720 and PD98059 (FIG. 5D). KT5720 did not affect ERK phosphorylation (FIG. 5E). Taken together, that YC-1 inhibits EZH2 expression is mediated by both PKA- and ERK-mediated pathways.

### Example 6

### YC-1 Inhibits EZH2 Through Src/Raf-1/ERK Pathway

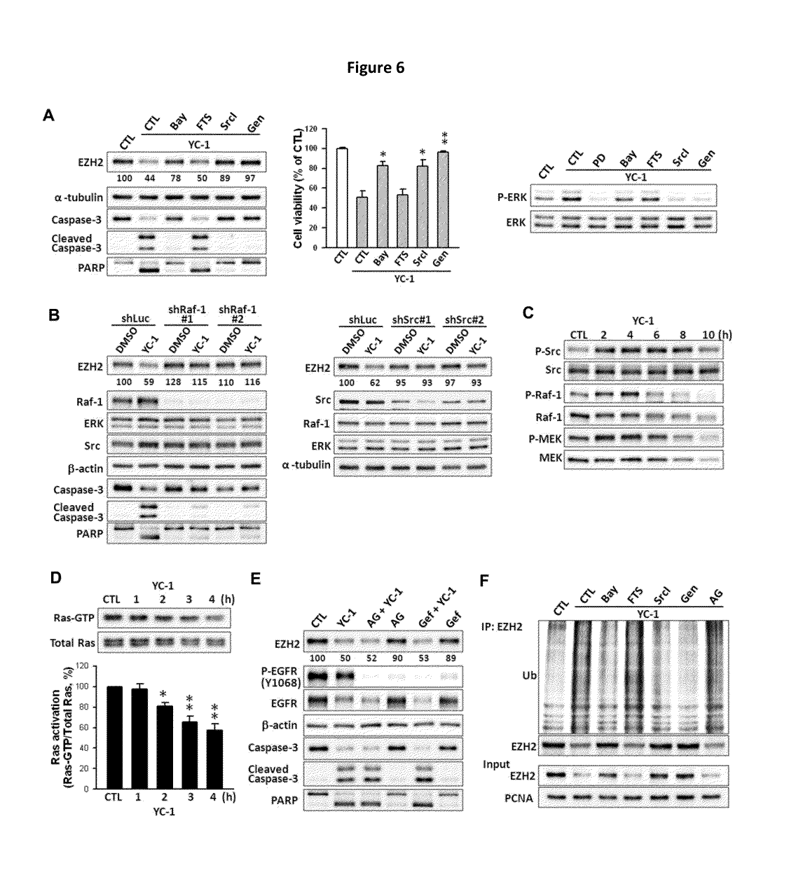

We next explored the upstream signaling molecules of ERK using the Raf-1 inhibitor (Bay-43-9006), Ras inhibitor (farnyesyl thiosalicylic acid), Src inhibitor (SrcI), and broad tyrosine kinase inhibitor (genistein). The inhibition of EZH2 levels and cell viability were attenuated by Bay-43-9006, SrcI, and genistein (FIG. 6A). Depletion of Raf-1 and Src markedly modulated YC-1-induced inhibition in both EZH2 expression and cell viability (FIG. 6B). Treatment of YC-1 caused a time-dependent phosphorylated activation of Src, Raf-1, and MEK (FIG. 6C). Surprisingly, farnyesyl thiosalicylic acid had no effect on YC-1-induced EZH2 inhibition. Another Ras inhibitor, manumycin A, also failed to influent EZH2-inhibition by YC-1 (data not shown). Furthermore, YC-1 could not induce Ras activation in MDA-MB-468 cells (FIG. 6D). MDA-MB-468 is characterized as EGFR predominant breast cancer cells (Clin Cancer Res 2004, 10:5367-74), and previous study revealed that YC-1 can inhibit EGFR expression on nasopharyngeal carcinoma (Biochem Pharmacol 2010, 79:842-52). We examined the role of EGFR in YC-1-inhibited EZH2 expression. Data showed that YC-1 rapidly induced EGFR phosphorylation and caused a significant decrease in EGFR protein expression after 6 h treatment (FIG. 11). However, the protein levels of EZH2 were not affected by two EGFR inhibitors, AG1478 and gefitinib (FIG. 6E). And, Bay-43-9006, SrcI, and genistein, not AG1478, significantly suppressed YC-1-induced EZH2 ubiquitination (FIG. 6F).

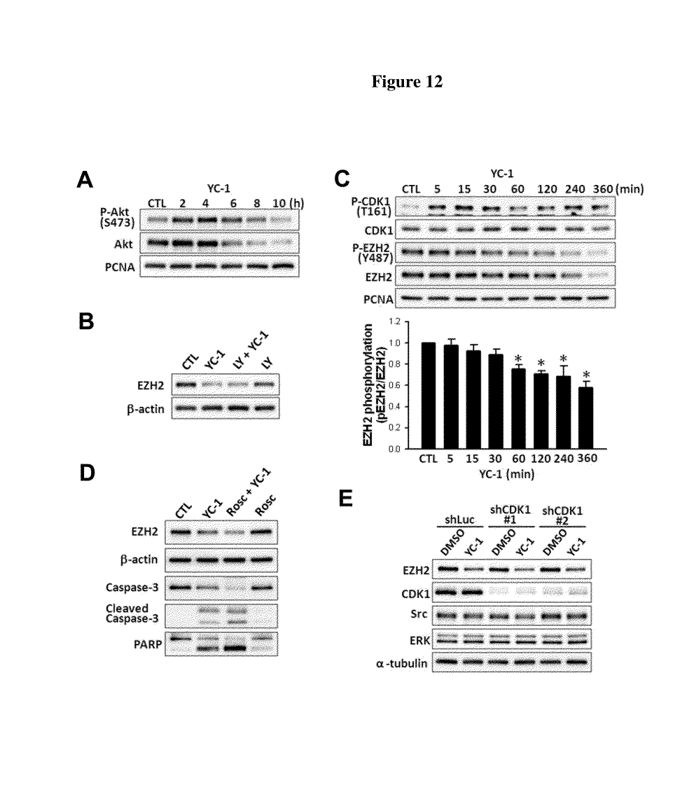

It has been demonstrated that the activation of Akt and CDK1 is associated with EZH2 inhibition and degradation (Science 2005, 310:306-10; Nat Cell Biol 2011, 13:87-94; J Biol Chem 2011, 286:28511-9). We found that Akt was activated by YC-1 (FIG. 12A). However, LY294002, a PI3K inhibitor, was unable to reverse YC-1-induced EZH2 suppression (FIG. 12B). YC-1 could induce the activation of CDK1, but no effect on the phosphorylation of EZH2 Tyr487 (FIG. 12C). Moreover, both CDK1 inhibitor, roscovitine, and specific CDK1 shRNA failed to modulate EZH2 protein levels and cell viability in response to YC-1 treatment (FIGS. 12D and 12E). These data suggested that YC-1 may mediate Src-Raf-1-MEK-ERK pathway to enhance EZH2 ubiquitination and its degradation.

### Example 7

### c-Cbl is Involved in YC-1 Downregulated EZH2 Expression

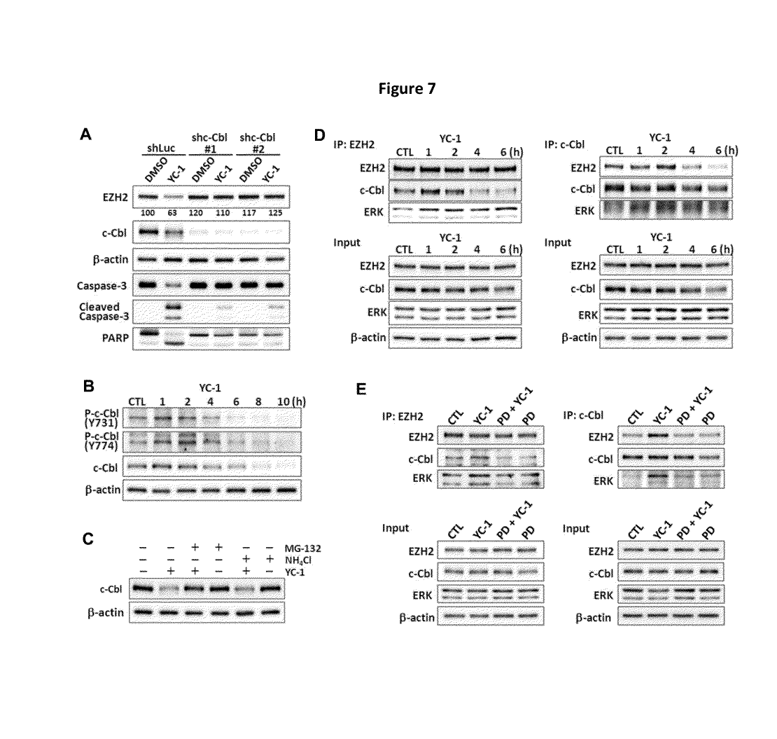

Next, we investigated which E3 ubiquitin ligases are responsible for YC-1-enhanced EZH2 degradation. PRAJA-1 has been identified and serves as EZH2 E3 ligase (Biochem Biophys Res Commun 2011, 408:393-8). We examined whether PRAJA-1 is associated with YC-1-induced EZH2 degradation event. However, due to the very low expression, PRAJA-1 was difficult to detect in MDA-MB-468 cells. Meanwhile, no effect in EZH2 expression was found while cells were treated with PRAJA-1 shRNA (data not shown). Our previous study found that Smurf2 acts as the E3 ubiquitin ligase which is responsible for the polyubiquitination and proteasome-mediated degradation of EZH2 during neuron differentiation (EMBO Mol Med 2013, 5:531-47). Therefore, whether Smurf2 mediates EZH2 degradation in response to YC-1 induction was also investigated. However, knockdown of Smurf2 by shRNA was unable to prevent the degradation of EZH2 protein (data not shown). There might be other ubiquitin ligases that are responsible for EZH2 degradation. Interestingly, we found that the suppression of EZH2 and apoptotic activation by YC-1 were almost completely abolished when c-Cbl was depleted (FIG. 7A). Previous study proved that c-Cbl mediates the ubiquitination and degradation of EGFR (J Biol Chem 2004, 279:37153-62). In this study, that c-Cbl Tyr731 and Tyr774 underwent rapid phosphorylation after 1 h YC-1 treatment was observed (FIG. 7B). The levels of c-Cbl protein expression were inhibited by YC-1 (FIG. 7B). This inhibition of c-Cbl could be reversed by MG-132, not NH₄Cl (FIG. 7C). Furthermore, YC-1 induced the complex formation of EZH2-c-Cbl-ERK after 1 h induction and reach maximum at 2 h (FIG. 7D), which coincided with c-Cbl phosphorylation. This complex could be disrupted by the treatment of MEK inhibitor, PD98059 (FIG. 7E). These data demonstrated that YC-1 leads to activation of c-Cbl followed by ERK activation, then complex forming with EZH2, resulting in EZH2 ubiquitination and proteasome degradation.

### CONCLUSIONS

EZH2 is overexpressed by cancer cells and functions as a tumor suppressor gene of epigenetic silencing, and its expression level is highly correlated to cancer metastasis ability. Here, we identified a new anticancer agent YC-1 in triple negative breast cancer cells, MDA-MB-468. Acting as an inhibitor of EZH2, a histone trimethyltransferase, YC-1 effectively inhibited cell viability and clonogenic ability and enhanced caspases activation on MDA-MB-468. Furthermore, YC-1 and YC-1-succinate reduced tumor in MDA-MB-468 xenograft mouse model. YC-1 concentration- and time-dependently downregulated the expression of EZH2 as well as other Polycomb repress complex members, including SUZ12, RbAp48, Ring1A, Ring1B, and Bmi1. Knockdown of EZH2 reduced the susceptibility of MDA-MB-468 cells to YC-1-induced apoptosis. Moreover, that suppression of EZH2 was found in tumor from YC-1-treated MDA-MB-468 xenograft mice. Proteasome inhibitor, MG-132, modulated YC-1-induced-EZH2 inhibition. Both degradation rate and ubiquitination of EZH2 protein were enhanced by YC-1. Down-regulation of EZH2 by YC-1 was associated with the activation of protein kinase A and Src-Raf-ERK-mediated pathways. And, depletion of c-Cbl, E3 ubquitin ligase, abolished YC-1-induced-EZH2 inhibition and apoptosis. YC-1 rapidly increased c-Cbl phosphorylation to induce signaling association with ERK and EZH2. A MEK inhibitor, PD98059, disrupted the interaction among EZH2, ERK, and c-Cbl. We discovered that YC-1 induces apoptosis and inhibition of tumor cell growth on MDA-MB-468 breast cancer cells through a down-regulation mechanism of EZH2 by activating c-Cbl and ERK. Following the same protocols as described in the examples, the prodrug YC-1-succinate is expected to have similar results and act by the same mechanism as YC-1.

What is claimed is:

1. A method for treating breast cancer, comprising administering to a subject suffering from breast cancer an effective amount of succinic acid mono-[5-(1-benzyl-1H-indazol-3-yl)-furan-2-ylmethyl]ester (YC-1-succinate) or a pharmaceutically acceptable salt thereof.

2. The method according to claim 1, wherein the breast cancer is triple negative breast cancer.

3. The method according to claim 1, wherein YC-1-succinate is administered orally.

4. The method according to claim 1, wherein YC-1-succinate is administered by intravenous injection.

5. A method for inhibiting the overexpression of EZH2 in breast cancer cells, comprising administering to the cancer cells an effective amount of YC-1 (3-(5′-hydroxymethyl-2′-furyl)-1-benzyl indazole), YC-1-succinate (succinic acid mono-[5-(1-benzyl-1H-indazol-3-yl)-furan-2-ylmethyl]ester), or a pharmaceutically acceptable salt thereof.

6. The method according to claim 5, wherein YC-1 is administered.

7. The method according to claim 5, wherein YC-1-succinate is administered.

8. The method according to claim 5, wherein the breast cancer cells are triple negative breast cancer cells.

9. The method according to claim 5, wherein the breast cancer cells are MDA-MB-468.


SEQUENCE LISTING (4 sequences)

SEQ ID NO: 1 - DNA, 23 bp, Homo sapiens
cgcttttcttctgtaggcgatgt

SEQ ID NO: 2 - DNA, 21 bp, Homo sapiens
tgggtgttgcatgaaaagaat

SEQ ID NO: 3 - DNA, 19 bp, Homo sapiens
agccacatcgctcagacac

SEQ ID NO: 4 - DNA, 18 bp, Homo sapiens
gcccaatacgaccaaatc